(19) United States
(12) Patent Application Publication
(10) Pub. No.: US 2013/0244336 A1
(43) Pub. Date: Sep. 19, 2013

(54) ODOUR AND/OR GAS IDENTIFICATION SYSTEM

(71) Applicant: Sensirion AG, Stafa (CH)

(72) Inventors: Felix MAYER, Stafa (CH); Markus GRAF, Zurich (CH)

(21) Appl. No.: 13/798,377

(22) Filed: Mar. 13, 2013

(30) Foreign Application Priority Data
Mar. 15, 2012 (EP) 12001781.9

(51) Int. Cl. G01N 33/00

(52) U.S. Cl. CPC G01N 33/0004
USPC 436/147; 702/27; 422/83; 436/149

(57) ABSTRACT

A method for identifying an odour and/or gas, an odour and/or gas is measured with a portable electronic device comprising a chemical sensor, the chemical sensor being sensitive to different analytes. A measurement tuple is supplied which comprises a set of tuple elements with each tuple element of the set of tuple elements providing a value measured by a dedicated cell of the chemical sensor and/or under a dedicated operating condition of the chemical sensor or of a cell of the chemical sensor. The measurement tuple is compared to one or more reference tuples with each reference tuple representing an odour and/or gas and comprising a set of tuple elements and an identifier for the odour and/or gas represented by the reference tuple. One or more odour and/or gas identifiers are returned to the portable electronic device subject to a result of the comparison.

### CROSS REFERENCE TO RELATED APPLICATIONS

This application claims the priority of European Patent Application 12 001 781.9, filed Mar. 15, 2012, the disclosure of which is incorporated herein by reference in its entirety.

### BACKGROUND OF THE INVENTION

The present invention relates to an odour and/or gas identification system and to a method for identifying an odour and/or a gas. In today's world of pervasive electronic devices, it would be desirable to get support in identifying an odour and/or gas.

### SUMMARY OF THE INVENTION

According to a first aspect of the present invention, an odour and/or gas identification system is provided comprising a portable electronic device comprising a chemical sensor being sensitive to different analytes and being adapted for supplying a measurement tuple comprising a set of tuple elements with each tuple element of the set of tuple elements providing a value measured during an odour and/or gas measurement by a dedicated cell of the chemical sensor and/or under a dedicated operating condition of the chemical sensor or of a cell of the chemical sensor. A database contains a set of reference tuples with each reference tuple representing an odour and/or gas and comprises a set of tuple elements with each tuple element of the set of tuple elements providing a value characteristic for being sensed by the dedicated cell of the chemical sensor and/or under the dedicated operating condition of the chemical sensor or of the cell of the chemical sensor in the presence of the odour and/or gas represented by the concerned reference tuple, and an identifier for the odour and/or gas represented by the concerned reference tuple. An evaluation unit is provided for comparing the measurement tuple to one or more reference tuples out of the set of reference tuples and subject to a result of the comparison returning one or more odour and/or gas identifiers to the portable electronic device. Preferred embodiments of the system may contain one or more of the following features:

the database and the evaluation unit are arranged remote from the portable electronic device, the electronic portable device is adapted to send the measurement tuple to the evaluation unit, and the evaluation unit is adapted to receive the measurement tuple from the electronic device and is adapted to send the one or more odour and/or gas identifiers to the portable electronic device;

the portable electronic device contains the evaluation unit, the database is arranged remote from the portable electronic device, and the electronic portable device is adapted to fetch one or more reference tuples from the database for comparing the measurement tuple to;

the database and the evaluation unit are contained in the portable electronic device;

the portable electronic device comprises a display for displaying the one or more odour and/or gas identifiers;

the chemical sensor comprises an array of sensor cells;

each sensor cell is mainly sensitive to a different one of the analytes the chemical sensor is sensitive to;

the portable electronic device comprises at least one of a temperature sensor for compensating temperature dependent signal variations in a signal of the chemical sensor, and a humidity sensor for compensating humidity dependent signal variations in a signal of the chemical sensor;

the portable electronic device comprises an input unit for triggering an odour and/or gas measurement;

the chemical sensor is adapted to continuously supply measurement tuples over time;

the portable electronic device comprises a selector unit for selecting one or more of a multitude of databases for applying reference tuples from;

the chemical sensor is adapted to supply the measurement tuple in combination with a chemical sensor identifier;

the chemical sensor is adapted to supply the measurement tuple in combination with a time stamp;

the chemical sensor is adapted to supply the measurement tuple in combination with location information indicating a current location of the portable electronic device;

the portable electronic device is one of a mobile phone, a handheld computer, an electronic reader, a tablet computer, a game controller, a pointing device, a photo or a video camera, and a computer peripheral.

According to another aspect of the present invention, a portable electronic device is provided for use in an odour and/or gas identification system according to any one of the previous embodiments.

According to a further aspect of the present invention, a method is provided for identifying an odour and/or gas. An odour and/or gas is measured with a portable electronic device comprising a chemical sensor unit, the chemical sensor unit being sensitive to different analytes. A measurement tuple is supplied comprising a set of tuple elements with each tuple element of the set of tuple elements providing a value measured by a dedicated cell of the chemical sensor and/or under a dedicated operating condition of the chemical sensor or of a cell of the chemical sensor during an odour and/or gas measurement. The measurement tuple is compared to one or more reference tuples with each reference tuple representing an odour and/or gas and comprising a set of tuple elements and an identifier for the odour and/or gas represented by the reference tuple. Each tuple element of the set of tuple elements provides a value characteristic for being sensed by the dedicated cell of the chemical sensor and/or under the dedicated operating condition of the chemical sensor or of the cell of the chemical sensor in the presence of the odour and/or gas represented by the concerned reference tuple. Subject to a result of the comparison one or more odour and/or gas identifiers are returned to the portable electronic device.

Preferred embodiments of the method may contain one or more of the following features:

the measurement tuple is compared to the one or more reference tuples by means of determining a deviation between measurement tuple elements and counterpart reference tuple elements with a measurement tuple element and its counterpart reference tuple element being assigned to the same cell of the chemical sensor and/or to the same operating condition of the chemical sensor or of the cell of the chemical sensor, and the identifier of a reference tuple compared to the measurement tuple is returned subject to the deviations determined;

the tuple elements to be compared comprise normalized values;

each deviation is compared to a threshold assigned, and the identifier of a reference tuple is compared to the measurement tuple is returned in case a number n of deviations is below the assigned thresholds.

According to another aspect of the present invention, a computer program medium comprising computer program code means is provided for implementing the following steps when executed on a processing unit: A measurement tuple is received from a chemical sensor unit and comprises a set of tuple elements with each tuple element of the set of tuple elements provides a value measured by a dedicated cell of the chemical sensor and/or under a dedicated operating condition of the chemical sensor or of a cell of the chemical sensor during an odour and/or gas measurement. The measurement tuple is compared to one or more reference tuples with each reference tuple representing an odour and/or gas and comprising a set of tuple elements and an identifier for the odour and/or gas represented by the reference tuple. Each tuple element of the set of tuple elements provides a value characteristic for being sensed by the dedicated cell of the chemical sensor and/or under the dedicated operating condition of the chemical sensor or of the cell of the chemical sensor in the presence of the odour and/or gas represented by the concerned reference tuple. Subject to a result of the comparison one or more odour and/or gas identifiers are returned.

Other advantageous embodiments are listed in the dependent claims as well as in the description below.

All described embodiments similarly pertain to the system, the method, and the computer program medium. Synergetic effects may arise from different combinations of the embodiments although they might not be described in detail.

### BRIEF DESCRIPTION OF THE DRAWINGS

The embodiments defined above and further aspects, features and advantages of the present invention can also be derived from the examples of embodiments to be described hereinafter and are explained with reference to the drawings. In the drawings the figures illustrate in

FIG. 1 a usage scenario with a mobile phone according to an embodiment of the present invention,

FIG. 2 a block diagram of a portable electronic device according to an embodiment of the present invention,

FIG. 3 a functional diagram of an odour and/or gas identification system according to an embodiment of the present invention,

FIG. 4 a flow diagram representing a method according to an embodiment of the present invention,

FIG. 5 a top view on a chemical sensor unit chip according to an embodiment of the present invention,

FIG. 6 an illustration of a comparison step in a method according to an embodiment of the present invention, and

FIG. 7 an illustration of two different measurement tuples as used in an embodiment of the present invention.

### DETAILED DESCRIPTION OF EMBODIMENTS OF THE INVENTION

First it is referred to general aspects of embodiments of the present invention.

The odour and/or gas identification system comprises a portable electronic device, a database, and an evaluation unit.

The portable electronic device comprises a chemical sensor which is sensitive to different chemical analytes. By means of such chemical sensor a gas in the environment of the portable electronic device may be investigated at least as to the absence or presence of the subject analytes the chemical sensor is sensitive to. The detection of chemical substances or compounds contained in such gas may be of interest to a user. The gas may contain substances or compounds that can be smelled by a human being and in such case the gas composition may be perceived as an odour, scent or flavour, and such gas would be denoted and understood by the human being by an identifier for the odour. Other gases may not contain substances that can be smelled by a human being or may contain substances that can be smelled, however, only at a very little degree such that a concentration is not sufficient for a human to smell. Still such gas may be denoted and understood by a human being by an identifier denoting a corresponding chemical formula or a corresponding name.

Analytes may include chemical substances and/or chemical compounds, and may specifically include one or more of, for example, CO2, NOX, ethanol, CO, ozone, ammonia, formaldehyde, or xylene without limitation. The chemical sensor is adapted to detect at least one property of at least two different analytes, and preferably is sensitive to five or more different analytes. Hence, the gas supplied to the chemical sensor may be analyzed by means of the chemical sensor as to if and which of the chemical substances or compounds the chemical sensor is sensitive to are present in the gas supplied. A combination of analytes detected in the gas supplied may suggest for a certain odour or for a certain gas. It is always subject to a design of the chemical sensor as to how many different analytes >1 and/or how many different properties of an analyte the chemical sensor is sensitive to.

The chemical sensor is capable of measuring one or more properties of multiple different analytes. Hence, the chemical sensor may be understood as a sensor device for detecting one or even more properties of more than one analyte. A property may, for example, be a concentration of an analyte in a gas which, for example, may be the air surrounding the device. Other properties may be, for example, chemical properties such as a binding energy of an analyte. Or, the chemical sensor may comprise at least sensor material, e.g. in form of a layer, an analyte may interact with. As a result, an electrical property of the sensor material may be modified upon interaction such as its electrical conductance, which principle preferably is applied in metal oxide chemical sensors, or an optical property of the sensor material may be modified such as its transmission rate, for example. Then, the electrical or optical property of a combination of the analyte and the sensor material is measured and allows a conclusion as to the analyte, such as by way of comparison to a property of the sensor material measured without the presence of the analyte. It is noted that for the different analytes the chemical sensor is sensitive to it is not required to always measure the same property per analyte. Different properties may be measured for different analytes.

Specifically, the chemical sensor may be embodied as a sensor array. A sensor array may comprise a set of sensor cells, wherein each sensor cell may provide a layer of a material exhibiting different sensitivity such that each cell of the sensor array may specifically be mainly sensitive to a different analyte and as such may enable the portable electronic device to detect the presence or absence or concentration of such analyte. “Mainly” in this context shall mean that a sensor cell is more sensitive to the subject analyte than to other analytes. In other variants, each sensor cell may provide a sensor material, e.g. in form of a layer and also denoted as sensitive layer, an analyte may interact with. As a result of the interaction, which e.g. may be a catalytic reaction, an electrical property of the sensor material may be modified, such as its electrical conductance, which principle preferably is applied in metal oxide chemical sensors, or an optical property may be modified such as its transmission rate, for example. However, a sensor cell of such sensor array may in one embodiment exhibit not only sensitivity to its main analyte, but also to analytes other than the main analyte since such sensor cell may exhibit a cross-sensitivity to one or more analytes possibly representing main analytes for other cells. In another embodiment, the chemical sensor may be a single sensor cell, e.g. with a single layer, which however, may be sensitive to multiple different analytes. Such single cell may, in one embodiment, be sensitive to different analytes only under different operating conditions. For example, the sensor cell may mainly be sensitive to a first analyte x when being heated to a first temperature tx, and may mainly be sensitive to a second analyte y when being heated to a second temperature ty which is different from the first temperature tx. In another variant, a sensor array may comprise multiple sensor cells wherein at least one of the multiple sensor cells—and in another variant preferably all of the multiple sensor cells—is/are designed such that such cell/s may mainly be sensitive to different analytes under different operating conditions such as under different temperatures. In such specific embodiment, each of such cell/s may be provided with an individual heater. In other embodiments, all cells may be heated by the same heater.

However, the chemical sensor may be based on one of the following measurement principles without limitation: A chemomechanical principle, in which a mass change upon absorption is transformed into a surface acoustic wave, or into a cantilever resonance, for example. Alternatively, there may be thermal sensing concepts applied, e.g. by making use of pellistors which may serve as a catalytic thermal sensor in which heat is generated or consumed during combustion. Alternatively, the chemical sensor may rely on optical detection, such as in form of a microspectrometer, or an NDIR, or may make use of electrochemical reactions such as being enabled by solid state electrolytes in combination with voltammetric, potentiometric, or conductometric measurement principles. Chemiresistors may also be used, such as conducting and carbon-loaded polymers, preferably in a low-temperature arena, or, metal-oxide sensors such as tin oxide, tungsten oxide, gallium oxide, indium oxide, zinc oxide, titanium oxide, which preferably may be applied in a high-temperature arena. ISFET (ion-selective FET) may also be used, as well as chemocapacitors wherein it is preferred to use a polymer as active material.

In case the chemical sensor is embodied as a sensor array, the individual sensor cells may preferably be embodied as discrete sensor cells arranged on a common conductor board. In a different embodiment, the sensor cells may be represented by multiple chips the sensing structures are integrated in. Here, each individual chip may be packaged, i.e. encapsulated, and arranged on a conductor board. In an alternative arrangement, such multiple sensor chips may comprise a common package, such that these chips are encapsulated by a common encapsulation, which package finally is arranged on the conductor board. In a further embodiment, the sensor cells are monolithically integrated into a common sensor chip with a common substrate for all sensor cells. Such monolithic sensor chip may still be encapsulated and be arranged on and electrically connected to a conductor board of the portable electronic device.

Calibration data may be stored in a non-volatile memory of the chemical sensor. Such calibration data may be applied to the sensor signal for compensating for drifts in the sensor signal, for example.

The chemical sensor may be adapted to supply a measurement tuple comprising a set of tuple elements with each tuple element of the set of tuple elements being assigned to a dedicated cell of the chemical sensor and/or to a dedicated operating condition of the chemical sensor or of a cell of the chemical sensor. In the measurement tuple, after a measurement, each tuple element may hold a value measured with respect to the assigned sensor cell and/or the assigned operating condition during an odour and/or gas measurement. In case, each sensor cell and/or each operating condition is embodied for being mainly sensitive to an analyte assigned, each measurement tuple may also be interpreted as an indicator of the presence and possibly a concentration of such analyte. This means that the chemical sensor provides a measurement result in form of a tuple with each tuple element representing the result of an individual sensor cell in case of a sensor array which sensor cell is mainly sensitive to one of the analytes. Hence, the measurement result of the chemical sensor with respect to a measurement—i.e. a measurement taken simultaneously by all sensor cells or taken within a reasonable time frame for belonging to the same measurement scenario—is assembled in a data structure referred to as measurement tuple and is supplied to an evaluation unit as will be explained later on.

Preferably, each measurement tuple element provides a normalized value. This means, that values measured by the cells, for example, are normalized with respect to a norm. Such norm may be, for example, the value measured by one of the cells. Accordingly, each value measured is divided by the value measured by one of the sensor cells. Given that the value of each measurement is dependent on a magnitude/strength of the gas or odour, such magnitude dependence may not be preferred in comparing the measurement tuple to the one or more reference tuples as will be explained later on. In an application for identifying an odour, for example, comparing a strong dose of the subject odour as measured to a smaller dose of the subject odour as a reference would not lead to a match in the comparison and as such not lead to the desired result. Hence, it is preferred that all tuple elements, be it the ones of the measurement tuple and be it the ones of the one or more reference tuples contain normalized values and all make use of a common norm. Then, a tuple may also be considered as a normalized vector.

It is preferred that the chemical sensor may be assigned a processing unit for supporting the assembly of the measurement tuple. Such processing unit may be a processing unit dedicated to the chemical sensor unit, or may be a processing unit of the portable electronic device that is made available for the subject task.

Preferably, the measurement tuple is supplied in combination with one or more of the following data: a chemical sensor unit identifier, a time stamp, global positioning information referring to the location of the portable electronic device at the time of measurement, etc. With such additional information, the odour and/or gas identification request may be better tracked.

In a preferred embodiment, the measurement tuple is supplied in combination with location information. The current location information, e.g. embodied as GPS (global positioning system) information, may be supplied by a GPS sensor of the portable electronic device and may allow the evaluation unit to take known or assumed gas and/or odour conditions for a particular location into account in the evaluation of the measurement tuple. For example, NOx levels are expected to be larger in the vicinity of streets. Hence, in a preferred embodiment, the evaluation unit may determine an NOx concentration and returns an alarm in case the determined NOx concentration exceeds an NOx alarm trigger value. Preferably, the NOx alarm trigger value is made dependent on the location information, and specifically is increased or reduced with respect to a default value if the current location is close to a street. The likewise mechanism may apply to the determination of sulfuric compounds. Any alarm trigger value for a concentration of sulphuric compounds may be made location dependent, and specifically may be made location dependent if the current location is in a volcanic active region. Summarizing, the evaluation unit may in one embodiment adjust a trigger value to be compared to a concentration of an identified compound dependent on the current location information. In another embodiment, the evaluation unit may preselect one or more reference tuples to compare the measurement tuple with for identification purposes dependent on the location information. For example, if the current location is identified as being in an area of plants emitting odorous compounds [food industry, agriculture, land fill, . . . ], the measurement tuple may first be correlated by known odorous compounds for this area.

In another preferred embodiment, the measurement tuple is supplied in combination with a chemical sensor unit identifier. The chemical sensor unit identifier may be an identifier such as a serial number assigned to the chemical sensor unit by the manufacturer and may be stored in a memory of or otherwise assigned to the chemical sensor unit. In such embodiment, the evaluation unit may take the particular chemical sensor unit identifier into account in the evaluation of the measurement tuple. For example, different kinds of chemical sensors may require different interpretation of the measurement tuple when comparing the measurement tuple with the reference tuples. In one embodiment, in a sensor array of a first kind the sensor cells may be arranged in a different order than in a sensor array of a second kind and the reference tuples may have been measured by the first kind of the sensor array. Hence, the chemical sensor unit identifier may help in translating the measurement tuple supplied by the second kind of sensor array into a tuple as if supplied by the first kind of sensor array. In another example, different manufacturing lots of the same type of chemical sensor may preferably require different interpretation of the measurement tuple or different calibration data applied owed to manufacturing tolerances, for example. Especially, if the calibration data for the chemical sensor unit is not stored on the mobile device but remote, the remote (evaluation) unit may be enabled to access the suitable calibration data/curves to be applied to the measurement tuple. In another example, different manufacturing lots may exhibit different drift effects in the sensor signal. Hence, the evaluation unit may by means of the chemical sensor unit identifier apply lot specific drift compensation values which may be stored in a database. The compensation values may be gathered from measurements at the supplier or may be derived from an average drift field data. In another embodiment, the evaluation unit determines if the identifier of the chemical sensor unit providing the measurement tuple is a valid chemical sensor unit. For this purpose, the evaluation unit may have access to a database of registered chemical sensor unit identifiers, for example. In case, there is no match identified in this comparison step, the evaluation unit may return a message that the chemical sensor unit is not reliable, and/or may not perform an identification of the measurement tuple.

In another preferred embodiment, the measurement tuple is supplied in combination with a time stamp. The time stamp may be supplied by the internal timing function of the portable electronic device. In one embodiment, the evaluation unit may take the time into account in the evaluation of the measurement tuple. For example, the evaluation of the measurement at time n may depend on the evaluation of the measurement at time n-1. Hence, a history of measurements may be taken into account. In another embodiment, the evaluation unit may correct the measurement tuple for a time dependent drift, e.g. for a drift that is dependent on the operational time of the chemical sensor unit so far.

Next to an impact the additional data may have on the evaluation, one or more of the additional data may be logged together with the measurement tuple, and possibly the result of the evaluation for future tracking purposes.

A tuple in general may also be referred to as vector.

The database is a database containing a set of reference tuples with each reference tuple representing an odour and/or gas and comprising a set of tuple elements and an identifier for the odour and/or gas represented by the concerned reference tuple. For a given reference tuple its elements are assigned to dedicated cells of the chemical sensor and/or dedicated operating conditions of the chemical sensor or of the cell of the chemical sensor, and as a result possibly to the various analytes the chemical sensor mainly is sensitive to. And each tuple element holds a value characteristic for being sensed by the dedicated cell of the chemical sensor and/or under the dedicated operating condition of the chemical sensor or of the cell of the chemical sensor in the presence of the odour and/or gas represented by the concerned reference tuple. Such value may preferably a normalized value. Hence, a reference tuple represents an odour and/or gas and specifically defines an odour and/or gas with respect to the set of analytes the chemical sensor is sensitive to. For example, if the chemical sensor is mainly sensitive to nine different analytes, a reference tuple representing the odour of a “tulip” may contain nine tuple elements with each tuple element providing a value reflecting the presence, absence, or partial presence of the assigned analyte out of the nine analytes the chemical sensor mainly is sensitive to. Each reference tuple is tagged by an identifier identifying the odour and/or gas which the subject reference tuple represents. The identifyer may be embodied as a numeral identifier which, for example, may generally be used in a classification system for odours and/or gases. Or it may be a verbal descriptor of the odour, such as “rose”, for example. Or it may be any other kind of identifier for identifying odour and/or gases.

The database containing reference tuples for odours and/or gases may be built by a database provider who may have measured odours and/or gases by means of a portable electronic device of the same kind, i.e. preferably containing an identical chemical sensor as is used for measuring odours and/or gas and assembling the associate measurement tuple later on by a user of the portable electronic device, and as such being sensitive to the same analytes, and preferably showing the same sensitivity for each analyte. Each odour and/or gas measured may be flagged by the provider with an identifier. For example, the provider may hold the portable electronic device close to a rose, trigger a measurement, have the measurement values stored in form of a tuple and may have assigned, for example, via an input on a keyboard or a touchscreen of the portable electronic device a written description of the odour and/or gas as identifier. The measurement tuple and the assigned identifier may be transmitted to the database and be stored there, e.g. via a wireless link in case the portable electronic device has a wireless communication capability. In a different scenario, users, and in another embodiment users registered with the database provider, may add reference tuples to the database by this enhancing the database to a powerful source of odour and/or gas reference tuples.

The underlying idea is to support a user of a portable electronic device in identifying an odour and/or gas that the user possibly may smell and/or a chemical composition that the user may not smell and which is measured by the chemical sensor of the users portable electronic device. For this purpose, the measurement tuple may be compared to one or more reference tuples from the database for identifying the odour and/or gas measured. In case, for example, the odour measured is the odour of a rose, a comparison of the measurement tuple with reference tuples from the data base may more or less match with one of the reference tuples which reference tuple represents the odour of a rose and which reference tuple accordingly is tagged as “rose”. This tag/identifier then is returned as odour identified by the evaluation unit based on a comparison of the measurement tuple with one or more reference tuples of the database.

The comparison in the evaluation unit may include a determination of a deviation between each measurement tuple element and its counterpart reference tuple element. In this context, a measurement tuple element and its counterpart reference tuple element are linked in that they are assigned to the same cell of the chemical sensor and/or to the same operating condition of the chemical sensor or of the cell of the chemical sensor. Hence, both tuple elements indicate a value, an in particular a normalized value, of e.g. the absence, presence or partial presence of the assigned analyte which is measured on the one hand, and which is provided within the reference tuple for characterizing the presence of the assigned analyte in the subject odour on the other hand. Since a tuple generally contains at least two tuple elements, there may be at least two deviations determined in the evaluation process. In case normalization is applied to the values of the measurement and reference tuple elements, at least three tuple elements are required for allowing comparison of at least two of the tuple elements of the measurement and the reference tuple since one of the tuple elements is used for normalization. Hence, it may depend on a result of the at least two deviations if or if not an odour and/or gas identifier is returned. For example, each deviation may be compared to an assigned threshold, and the identifier of the reference tuple under investigation is returned in case a number n of deviations is below the assigned thresholds. This means that at least n analytes need to closely match a typical concentration of the subject analytes as is manifested in the subject reference tuple for the subject odour/gas.

In a variant, the identifier of two or more measurement tuples may be returned. This may be the case, if no single reference tuple unambiguously matches the measurement tuple. In such case, the two or more reference tuples coming closest to the measurement tuple are selected for an odour and/or gas suggestion. For example, the identifiers of n reference tuples with a lowest accumulation value of all tuple element deviations may be returned. In another embodiment, a statement as to a likelihood of the different odours and/or gases suggested may be returned in combination with the identifiers. In another scenario, a void identifier may be returned in case no single reference tuple has sufficiently matched the measurement tuple.

Preferably, the evaluation unit may apply some sort of pattern recognition for comparing the measurement tuple to one or more reference tuples. In this context, but also in a general context, a comparison between tuples and/or graphical representations of the tuples may include a comparison of a deviation between the measurement tuple and a reference tuple to a variable threshold, or, in another embodiment, to a threshold corridor/range within which the measurement tuple is expected to fall for compliance. Preferably all the reference tuples are compared to the measurement tuple in order to increase chances of a match. Smart algorithms may be applied for performing the comparison step. For example, only one measurement tuple element may be compared to all corresponding reference tuple elements. In a next step, only the x reference tuples containing the x reference tuple elements that came closest to the first measurement tuple element are selected for a comparison with the second measurement tuple element.

With respect to the allocation of components of the odour and/or gas identification system, the following embodiments are preferred:

In a first embodiment, the database and the evaluation unit are arranged remote from the portable electronic device. In view of the database claiming considerable storage space a server/a server system/a cloud system may preferably be used for providing the database. The evaluation unit may be represented by processing power at the same system remote from the portable electronic device or on a different system remote from the portable electronic device. A service provider may offer services to users or subscribers of identifying odours and/or gases received in form of measurement tuples. A user may register with the provider and receive the odour and/or gas identifiers in return for a measurement tuple sent. Any transmission of a measurement tuple and/or of an identifier return may be performed via a wireless link, e.g. based on a UMTS or a GPRS standard. In another embodiment, the provider may return the identifier via SMS.

In a different embodiment, the database is remote from the portable electronic device, however, the evaluation unit is arranged in the portable electronic device. The evaluation unit may be embodied as hardware or firmware or as software on a general purpose processor unit of the portable electronic device. In this scenario, the electronic portable device may request one or more reference tuples from the database for comparing the measurement tuple to locally, i.e. on the portable electronic device. Again, a wireless link may be used for requesting/fetching and receiving reference tuples from the database.

In a third embodiment, the database and the evaluation unit are arranged in the portable electronic device. Given that the storage space in portable electronic devices such as in smartphones has considerably grown over the previous years, a local storage of the entire database may be envisaged, too. In this embodiment, the chemical sensor, the evaluation unit and the tuple database all reside in the portable electronic device. Identifiers are returned within the portable electronic device and may be displayed on a display of the portable electronic device. The database may be updated regularly by a service provider, as may be the evaluation algorithm.

Hence, any portable electronic device such as a mobile phone, and in particular a smart phone, a handheld computer, an electronic reader, a tablet computer, a game controller, a pointing device, a photo or a video camera, or a computer peripheral—which listing is not limited—may in addition to its original function support the chemical and/or odour and/or gas identification as to its environment. Such portable electronic device as a result may primarily be designed for computing and/or telecommunication and/or other tasks in the IT arena, and now may be enhanced by the function of providing chemical information as to its environment. The user may learn about chemical substances, compositions and/or odours present in the devices surroundings, and may use, transmit or else further analyse such information. For the reason that such portable electronic device typically includes interfaces to a remote infrastructure, such information may also be transmitted elsewhere and used elsewhere. In an alternative, the user himself/herself may benefit from the information provided by the chemical sensor in that actions may be taken in response to detected gases and/or odours, including but not limited to toxic substances and compounds such as CO. An alarm may be triggered once such compound is identified in form of a return of the “CO” identifier, for example. Other measurements may be taken out of pure interest, such as, for example, the measurement of the odour of a flower or the odour of a drink. In response to such measurement, the identifier/name of an odour unknown to the user, such as “sunflower”, for example, or “bourbon 10 yrs old”, may be displayed to the user.

In another embodiment, there may be at least one additional sensor provided out of a humidity sensor and a temperature sensor. These sensors may help in compensating temperature induced and/or humidity induced signal variations in a signal of the chemical sensor. Preferably the temperature sensor and/or the humidity sensor may be arranged in proximity to the chemical sensor, for example in the same opening of a housing of the portable electronic device.

In a preferred embodiment, the portable electronic device comprises an input unit for triggering an odour and/or gas measurement. Such input may be a key or a touchscreen element, for example. It may enable the user to start a measurement. The user may first arrange the portable electronic device close to a source of odour and/or gas and then trigger the measurement. In a different embodiment the chemical sensor unit is adapted to periodically supply measurement tuples over time, for example at regular intervals. This operational mode may be advantageous when the user may stay in a polluted environment, for example.

In general, any measurement finalized by the chemical sensor unit may initiate a transfer of the measurement tuple to the evaluation unit and may initiate an evaluation there.

In a preferred embodiment, there may be multiple databases provided by different providers or users, and the portable electronic device may comprise a selector unit for offering a selection of one or more of a multitude of databases to the user for applying reference tuples from. The selector unit may be built in form of a key or a touchscreen, for example. There may be scenarios in which official or commercial provider databases may be trusted more than private user databases such a selection is desirable. This may be true, for example, for databases containing reference vectors representing vapours of organic solvents. An associate service provider may have collected and tagged the signal vectors under controlled conditions.

Same or similar elements are referred to by the same reference numerals across all Figures.

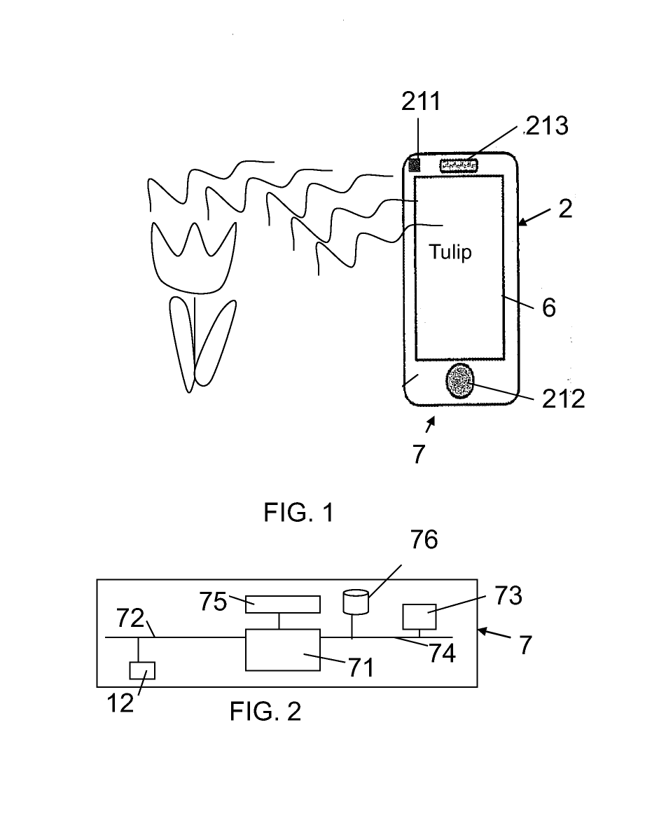

FIG. 1 illustrates a usage scenario with a mobile phone 7 according to an embodiment of the present invention. Apart from a standard microphone as an input device which microphone is arranged in an opening 212 of a front wall 21 of a housing 2, a chemical sensor is arranged in another opening 211 of the front wall 21, which opening 211 is arranged in proximity to yet another opening 213 for a standard speaker of the mobile phone 7. The mobile phone 7 is held close to bunch of tulips which emit a tulip specific odour. This odour, and its chemical substances respectively, is sensed by the chemical sensor. By means of a database and an evaluation unit—which may, in this example, be arranged in the mobile phone 7 itself—the sensed odour is compared to reference odours and is finally identified as an odour from tulips. This result is displayed on a display 6 of the mobile phone 7.

FIG. 2 shows a schematic hardware oriented block diagram of a portable electronic device in form of a mobile phone 7. A microprocessor 71 is connected via electrical conductors 72 to a chemical sensor 12, which electrical conductors 72 specifically may be conductors of a flexible printed circuit board. The chemical sensor 12 contains signal processing capability in order to output an odour and/or gas tuple measured. A routine for analysing the odour supplied by the chemical sensor 12 in form of a measurement tuple may be executed by an evaluation unit. A hardware of the evaluation unit may be represented by the microprocessor 71, and a software of the evaluation unit may be represented by a program element stored in a memory 73 connected to the microprocessor 71 via a bus system 74. A database 76 containing reference tuple data may be connected to the microprocessor 71 via the bus system 74, too. A wireless interface 75 may be connected to the microprocessor 71.

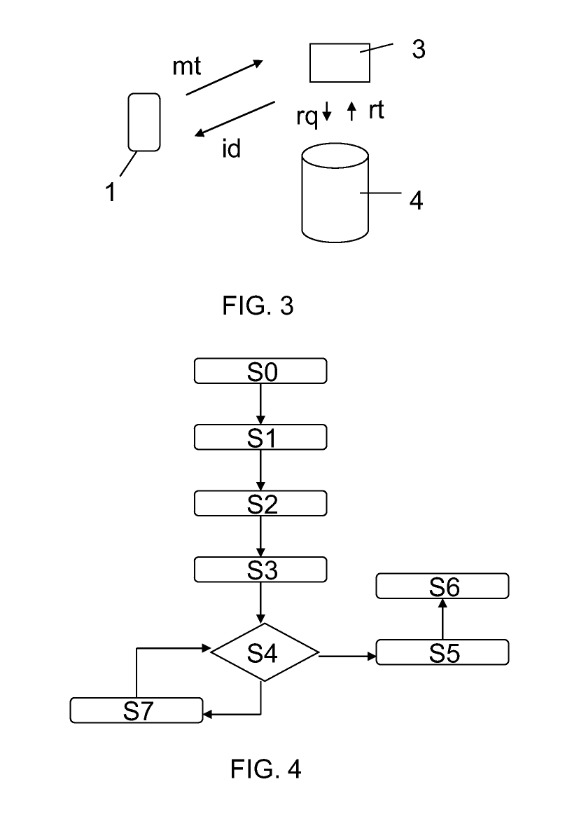

FIG. 3 illustrates a functional diagram of an odour and/or gas identification system according to an embodiment of the present invention. In this specific case, the portable electronic device may be embodied as a tablet computer 1 comprising a chemical sensor not further shown in FIG. 3. The chemical sensor provides a measurement tuple mt to an evaluation unit 3 remote from the tablet computer 1. The evaluation unit 3 may be embodied in a server hosting a service for odour and/or gas identification. Once the evaluation unit 3 has received a measurement tuple mt, this is taken as a request to start an identification process which is initiated by submitting a request rq to a database 4 for reference tuple data. The database 4 is remote from the tablet computer 1 and the evaluation unit 3 and contains storage space for storing reference tuples rt representing odours and/or gases, for example. The reference tuples rt are transmitted to the evaluation unit 3 as requested and are compared to the measurement tuple mt there. Once a match is identified between the measurement tuple mt and one of the reference tuples rt, an identifier id for the odour and/or gas represented by this specific reference tuple rt is returned to the tablet computer 1.

FIG. 4 illustrates a flow diagram representing a method according to an embodiment of the present invention. In a first step S0, a measurement of an odour and/or gas is initiated by a user of a portable electronic device containing a chemical sensor. In step S1, the measurement is performed, and the measurement results are assembled in a data structure denoted as measurement tuple and having a digital format. In step S2, the measurement tuple is sent to an evaluation unit located remote from the portable electronic device. In step S3, the evaluation unit sets a counter i to zero. In step S4, the evaluation unit which may together with a database reside in a server, retrieves the i^{th} reference tuple from the database and compares the measurement tuple to the i^{th} reference tuple. In case the measurement tuple matches the i^{th} reference tuple (Y), an identifier of the subject reference tuple is returned by the evaluation unit to the portable electronic device in step S5. In step S6, the identifier may be displayed on a display of the portable electronic device. In case the measurement tuple does not match the i^{th} reference tuple (N), the reference tuple counter i is increased in step S7, and in step S4 the measurement tuple is compared to the next reference tuple from the database.

FIG. 5 illustrates a top view on a chip 112 representing a chemical sensor as is used in an embodiment of the present invention. The chip 112 comprises a chemical sensor structure 1121 which takes the shape of a sensor array comprising multiple symbolic sensor cells 1122, in the present example, thirty six sensor cells 1122. In addition a humidity sensitive structure 1111 is arranged next to the chemical sensor structure 1121, and electronic circuitry 1123 is integrated into the chemical sensor chip 112 which electronic circuitry 112 is responsible for applying a humidity compensation of the signal supplied by the chemical sensor structure 1121, for linearizing and A/D converting the sensor signal, and for building the measurement tuple, for example.

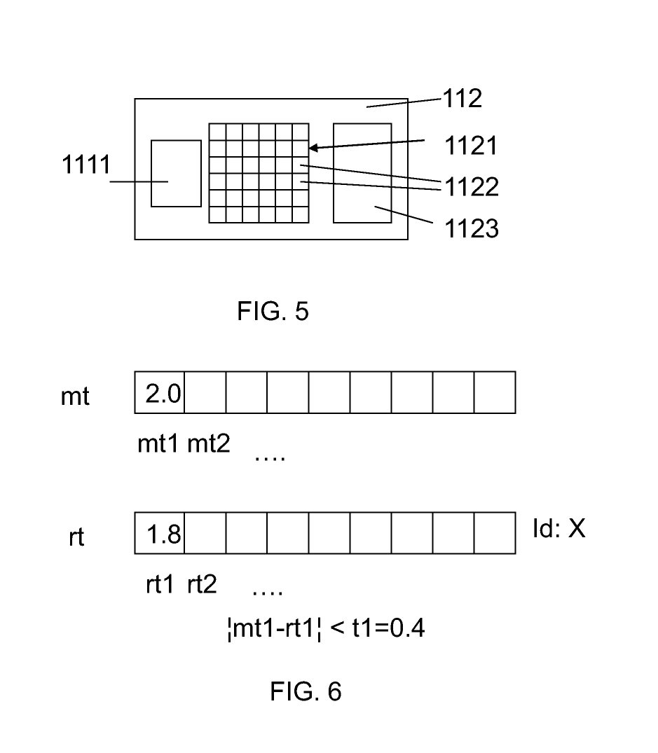

FIG. 6 illustrates an illustration of a comparison step in a method according to an embodiment of the present invention. A sample measurement tuple mt is depicted containing nine measurement tuple elements mt1 . . . mx . . . mt9, each tuple element holding a value wherein only the value 2.0 of the first measurement tuple element mt1 is shown for illustration purposes. A sample reference tuple rt is depicted containing nine reference tuple elements rt1 . . . rtx . . . rt9, each tuple element holding a value wherein only the value 1.8 of the first reference tuple element rt1 is shown for illustration purposes. The reference tuple rt represents odour id=X. The first measurement tuple element mt1 is assigned to a first sensor cell of the chemical sensor which first sensor cell is mainly sensitive to a first analyte. The corresponding first reference tuple element rt1 is assigned to the same sensor cell and hence, provides a value as to the same first analyte. The value of the first measured tuple element mt1 is compared to the first reference tuple element rt1 by determining a deviation |mt1−rt1| which deviation is compared to a first threshold t1, which threshold is the present example is set to 0.4. The interpretation of the foregoing may be as follows: A concentration of 2.0 of the first analyte was measured. The reference odour named “X” typically shows a concentration of this analyte of 1.8. As long as the deviation of the measured concentration from the reference concentration is less than 0.4, the measured odour and/or gas may be “X” subject to a concentration of the other analytes measured which concentrations may be compared to the reference concentrations in the same way.

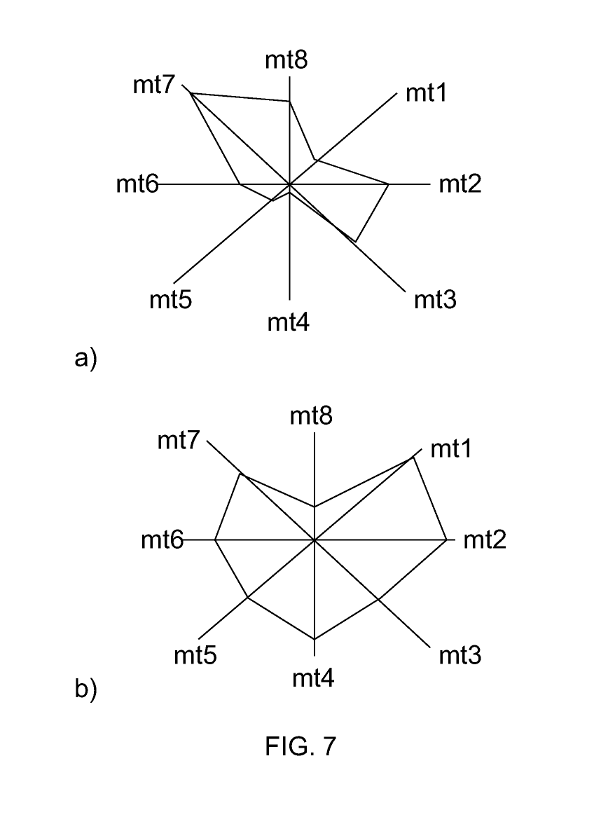

FIG. 7 illustrates in diagrams a) and b) two different measurement tuples mt as used in an embodiment of the present invention. Each measurement tuple mt is sensed by a chemical sensor being sensitive to eight analytes, such that a measurement tuple mt contains eight tuple elements mt1 . . . mt8. The measurement tuple mt according to diagram 7a) represents a measurement of a first gas/odour, and the measurement tuple mt according to diagram 7b) represents a measurement of a second gas/odour. Each measurement tuple mt is represented by a spider diagram with eight axes, and with the value of each tuple element mt1 . . . mt8 being reflected on the respective axis crossing. The values as shown may be normalized values in order for a specific odour to result in the same spider diagram every time independent of a strength of the odour. For example, each measured value may be converted into a normalized value by dividing the value by the value measured for the first sensor cell, i.e. mt1. Preferably, normalized patterns are compared to each other. Other approaches of normalizations may be applied, for example, a normalization with respect to an area of the spider diagram, etc. Such spider diagram measurement tuple representation may support graphical pattern recognition. The reference tuples may also be stored as graphical representations in form of spider diagrams. In a best match algorithm, the reference pattern that matches the measurement pattern best is identified and its tag is returned.

While there are shown and described presently preferred embodiments of the invention, it is to be distinctly understood that the invention is not limited thereto but may be otherwise variously embodied and practised within the scope of the following claims.

1. An odour and/or gas identification system, comprising a portable electronic device comprising a chemical sensor, wherein the sensor is sensitive to to different analytes and is adapted for supplying a measurement tuple, said measurement tuple comprising a set of tuple elements, and each tuple element of the set of tuple elements providing a value measured during at least one of an odour or gas measurement
wherein said measurement is made by a dedicated cell of the chemical sensor, or wherein said measurement is made under a dedicated operating condition of the chemical sensor or of a cell of the chemical sensor;
a database containing a set of reference tuples with each reference tuple representing at least one of an odour or gas and comprising
a set of tuple elements with each tuple element of the set of tuple elements providing a value characteristic sensible by the chemical sensor in the presence of at least one of the odour or gas represented by a corresponding reference tuple, and
an identifier for the odour and/or gas represented by the corresponding reference tuple;
an evaluation unit for comparing the measurement tuple to one or more reference tuples out of the set of reference tuples, and subject to a result of the comparison, returning at least one odour or gas identifier to the portable electronic device.

2. The system according to claim 1,
wherein the database and the evaluation unit are arranged remote from the portable electronic device,
wherein the electronic portable device is adapted to send the measurement tuple to the evaluation unit, and
wherein the evaluation unit is adapted to receive the measurement tuple from the electronic device and is adapted to send the at least one odour or gas identifiers identifier to the portable electronic device.

3. The system according to claim 1,
wherein the portable electronic device contains the evaluation unit,
wherein the database is arranged remote from the portable electronic device, and
wherein the electronic portable device is adapted to fetch one or more reference tuples from the database for comparing the measurement tuple to.

4. The system according to claim 1,
wherein the database and the evaluation unit are contained in the portable electronic device.

5. The system according to claim 1,
wherein the portable electronic device comprises a display for displaying the at least one odour or gas identifier.

6. The system according to claim 1,
wherein the chemical sensor comprises an array of sensor cells, and
wherein each sensor cell is sensitive to a different analyte.

7. The system according to claim 1,
wherein the portable electronic device comprises at least one of
a temperature sensor for compensating temperature dependent signal variations in a signal of the chemical sensor, and
a humidity sensor for compensating humidity dependent signal variations in a signal of the chemical sensor.

8. The system according to claim 1,
wherein the portable electronic device comprises an input unit for triggering at least one of an odour or gas measurement.

9. The system according to claim 1,
wherein the chemical sensor is adapted to continuously supply measurement tuples over time.

10. The system according to claim 1,
wherein the portable electronic device comprises a selector unit for selecting one or more of a multitude of databases for applying reference tuples from.

11. The system according to claim 1,
wherein the chemical sensor is adapted to supply the measurement tuple in combination with a chemical sensor identifier.

12. The system according to claim 1,
wherein the chemical sensor is adapted to supply the measurement tuple in combination with a time stamp.

13. The system according to claim 1,
wherein the chemical sensor is adapted to supply the measurement tuple in combination with location information indicating a current location of the portable electronic device.

14. The system according to claim 1,
wherein the portable electronic device is one of:
a mobile phone,
a handheld computer,
an electronic reader,
a tablet computer,
a game controller,
a pointing device,
a photo or a video camera, or
a computer peripheral.

15. A portable device for use in a an odour and/or gas identification system according to claim 1.

16. A method for identifying an odour and/or gas, comprising the steps of
Measuring at least one of an odour or gas with a portable electronic device comprising a chemical sensor, the chemical sensor being sensitive to different analytes;
supplying a measurement tuple comprising a set of tuple elements with each tuple element of the set of tuple elements providing a value measured during at least one of an odour or gas measurement
wherein said measurement is made by a dedicated cell of the chemical sensor, or wherein said measurement is made under a dedicated operating condition of the chemical sensor or of a cell of the chemical sensor;
comparing the measurement tuple to one or more reference tuples with each reference tuple representing at least one of an odour or gas and comprising
a set of tuple elements with each tuple element of the set of tuple elements providing a value characteristic sensible by the chemical sensor in the presence of the odour and/or gas represented by a corresponding reference tuple, and
an identifier for the odour or gas represented by the reference tuple; and
returning at least one odour or gas identifier to the portable electronic device subject to a result of the comparison.

17. The method according to claim 16, further comprising
comparing the measurement tuple to the one or more reference tuples by determining a deviation between measurement tuple elements and counterpart reference tuple elements, wherein a measurement tuple element and its counterpart reference tuple element are assigned to the same cell of the chemical sensor or are assigned to the same operating condition of the chemical sensor or of the cell of the chemical sensor,
wherein the identifier of a reference tuple compared to the measurement tuple is returned subject to the deviations determined.

18. The method according to claim 17, wherein the tuple elements to be compared comprise normalized values.

19. The method according to claim 17, further comprising
comparing each deviation to a threshold assigned,
wherein the identifier of a reference tuple compared to the measurement tuple is returned in case a number n of deviations is below the assigned thresholds.

20. A computer program medium comprising computer program code for implementing the following steps when executed on a processing unit:
comparing a measurement tuple received from a chemical sensor unit and comprising a set of tuple elements with each tuple element of the set of tuple elements providing a value measured by a dedicated cell of the chemical sensor and/or under a dedicated operating condition of the chemical sensor or of a cell of the chemical sensor during at least one of an odour or gas measurement, to one or more reference tuples with each reference tuple representing at least one of an odour or gas and comprising
a set of tuple elements with each tuple element of the set of tuple elements providing a value characteristic sensible by the chemical sensor in the presence of the odour or gas represented by the concerned reference tuple, and
an identifier for the odour or gas represented by the reference tuple; and
returning at least one odour or gas identifier subject to a result of the comparison.
